(19) United States
(12) Patent Application Publication
(10) Pub. No.: US 2021/0158902 A1
(43) Pub. Date: May 27, 2021

(54) SYSTEM AND METHOD FOR ALLELE INTERPRETATION USING A GRAPH-BASED REFERENCE GENOME

(71) Applicant: KONINKLIJKE PHILIPS N.V., EINDHOVEN (NL)

(72) Inventors: YONG MAO, Hawthorne, NY (US); KOSTYANTYN VOLYANSKYY, LARCHMONT, NY (US); NEVENKA DIMITROVA, PELHAM MANOR, NY (US)

(21) Appl. No.: 17/058,171

(22) PCT Filed: May 20, 2019
(86) PCT No.: PCT/EP2019/062905

§ 371 Date: Nov. 24, 2020

Related U.S. Application Data
(60) Provisional application No. 62/678,324, filed on May 31, 2018.

(51) Int. Cl. G16B 45/00; G16B 30/10; G16B 30/20

(52) U.S. Cl. CPC G16B 45/00; G16B 30/20; G16B 30/10

(57) ABSTRACT

A method (100) for generating a graph-based reference genome, comprising: (i) receiving (120) one or more older versions of a current reference genome, each comprising a plurality of nodes identifying the version of the reference genome and a location within that version for the respective node; (ii) aligning (130) each older version of the reference genome to the current reference genome to generate a graph-based reference genome, wherein the alignment is based on the location information; (iii) extracting (140), from a corpus of references, an allele and contextual information associated with the allele, wherein the respective reference identifies the version of the reference genome and a location of the allele within the version; and (iv) mapping (150) the allele and associated contextual information onto a node of the graph-based reference genome, based on the identified version of the reference genome and the location of the extracted allele within that version.

### FIELD OF THE DISCLOSURE

The present disclosure is directed generally to methods and systems for generating an annotated graph-based reference genome.

### BACKGROUND

Personal genomics is an increasingly important aspect of healthcare. Due to the emerging maturity of sequencing technology, new applications are continually proposed for personal genomic information. These new applications are typically aimed at identifying therapeutic options and/or tailoring therapeutic options to a particular patient based on the patient's personal profile comprising both genetic information (such as sequencing information, methylation, transcriptome, and/or other genetic/genomic information) and a clinical profile (such as age, gender, diagnosis, condition, history, and/or other clinical information).

Although obtaining a genomic profile is increasingly affordable, interpreting the results of a genomic profile is usually far more expensive due to the lack of available or accumulated knowledge. Since the first sequencers began to obtain genetic information, a very large corpus of medical literature has been generated to explain biomedical functions and mutation frequencies for many different populations. Although there is an enormous corpus of information, there is no simple or efficient methodology or framework to align the corpus of information.

For example, literature published in the early 2000s utilized an early version of the human reference genome, while recent publications may utilize a recent version such as GRCH37 or GRCH38. A mutation discussed in 2005 and in 2015 might correspond to different coordinates along different reference genomes. Accordingly, in order to interpret the function of a mutation or to prioritize mutations, it is normally a requirement that a researcher or clinician accumulate and review medical literature by hand. This is especially true when identifying causes of rare disease cases. If it were possible to accumulate literature and relevant references from all different versions of a reference genome around a specific phenotype or diagnosis, personalized medicine would be significantly enhanced.

A single, mono-ploidy or linear reference genome is a poor universal reference structure for a reference genome because it represents only a tiny fraction of variation and only for a period of time in which the specific version of the reference genome is utilized. To support changes made along a reference genome and looking forward to future versions of the genome, a graph-based reference genome provides a comprehensive framework to align knowledge at the level of alleles. A graph-based reference genome has the capability of integrating polymorphisms and mutations across populations and single individuals, among many other benefits.

### SUMMARY OF THE DISCLOSURE

There is a continued need for tools and methods that enable the collection and organization of literature regarding previous versions of a reference genome onto a current, graph-based version of the reference genome.

The present disclosure is directed to inventive methods and systems for generating an annotated graph-based reference genome. Various embodiments and implementations herein are directed to a system that enables reporting of allele and contextual information organized from a plurality of versions of a reference genome. The system aligns older versions of a reference genome onto a current version of the reference genome to create a graph-based reference genome. The graph-based reference genome includes nodes with information about the prior location of the nodes in the older versions of the reference genome. The system then extracts or receives information from the scientific literature about an allele and contextual information associated with that allele, including information about which old version of the reference genome the allele was identified in and the location of the allele in that old version of the reference genome. The extracted allele and contextual information is then mapped onto the graph-based reference genome by searching the graph-based reference genome for a node that comprises the extracted version of the reference genome and the extracted location.

Generally in one aspect, a method for generating an annotated graph-based reference genome is provided. The method includes: (i) receiving one or more versions of a reference genome, being older versions of a current reference genome, each of the one or more versions of the reference genome comprising a plurality of nodes, at least some of which comprise information identifying the version of the reference genome and a location within that version of the reference genome for the respective node; (ii) aligning each of the one or more received older versions of the reference genome to the current reference genome to generate a graph-based reference genome, wherein the alignment is based at least in part on the location information from the nodes of the received older version of the reference genome; (iii) extracting, from a corpus of references at least some of which each comprise information about an allele and contextual information associated with that allele, an allele and contextual information associated with the allele, wherein the respective reference identifies one of the one or more received older versions of the reference genome, and a location of the allele within the identified older version of the reference genome; and (iv) mapping the extracted allele and associated contextual information onto a node of the graph-based reference genome, based on the identified older version of the reference genome and the location of the extracted allele within that identified older version of the reference genome.

According to an embodiment, the method further comprises generating a report summarizing all the contextual information associated with a node of the graph-based reference genome; and providing, via a user interface, the generated report to a user.

According to an embodiment, the report comprises one or more of an allele frequency, appearance information, surrounding mutation information, and/or co-mutation rate.

According to an embodiment, mapping comprises annotating the node with the extracted allele and associated contextual information. According to an embodiment, mapping comprises annotating the node with an identification of the reference from which the allele was extracted.

According to an embodiment, the contextual information comprises information about a trait or medical condition associated with the allele. According to an embodiment, the contextual information comprises an identification of a reference from which the allele was identified or extracted. According to an embodiment, the contextual information comprises information about one or more people in which the allele was identified.

According to an embodiment, the method further comprises normalizing a plurality of alleles associated with a node of the graph-based reference genome.

According to another aspect is a system for generating an annotated graph-based reference genome. The system includes: (i) an alignment module configured to align each of a plurality of received older versions of a reference genome to a current reference genome to generate a graph-based reference genome, wherein the alignment is based at least in part on information from nodes of the received older version of the reference genome, at least some of the nodes comprising information identifying the version of the reference genome and a location within that version of the reference genome for the respective node; (ii) a mapping module configured to map a plurality of identified alleles onto one or more nodes of the graph-based reference genome based on the identified older version of the reference genome and the location of the extracted allele within that identified older version of the reference genome, wherein each of the plurality of identified alleles also comprises contextual information which is mapped onto the respective node with the respective allele; (iii) a reporting module configured to generate a report summarizing all the contextual information associated with a node of the graph-based reference genome; and (iv) a user interface configured to provide the generated report to a user.

According to an embodiment, the system further includes an extraction module configured to extract, from a corpus of references at least some of which each comprise information about an allele and contextual information associated with that allele, an allele and contextual information associated with the allele, wherein the respective reference identifies: (i) one of the one or more received older versions of the reference genome, and (ii) a location of the allele within the identified older version of the reference genome.

According to another aspect is a graph-based reference genome. The graph-based reference genome includes: (i) a plurality of annotated nodes of a current version of a reference genome, wherein each of the plurality of annotated nodes comprises information about an allele and contextual information associated with that allele from one or more prior versions of the reference genome, the contextual information comprising at least an identification of the prior version of the reference genome from which the allele was extracted and information about the genomic coordinates of the allele in the prior version of the reference genome from which the allele was extracted; and (ii) a plurality of edges, each connecting two nodes via a first or second end of each of said two nodes.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the various embodiments discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for generating an annotated graph-based reference genome, in accordance with an embodiment.

FIG. 2 is a schematic representation of a system for generating an annotated graph-based reference genome, in accordance with an embodiment.

FIG. 3 is a schematic representation of an annotated graph-based reference genome, in accordance with an embodiment.

### DETAILED DESCRIPTION OF EMBODIMENTS

The present disclosure describes various embodiments of a system and method for generating an annotated graph-based reference genome. More generally, Applicant has recognized and appreciated that it would be beneficial to provide a system for reporting allele and contextual information organized from a plurality of versions of a reference genome. The system aligns older versions of a reference genome onto a current version of the reference genome to create a graph-based reference genome. The system extracts or receives information from the scientific literature about an allele and contextual information associated with that allele, including information about which old version of the reference genome the allele was identified in and the location ofthe allele in that old version of the reference genome. The extracted allele and contextual information is then mapped onto the graph-based reference genome by searching the graph-based reference genome for a node that comprises the extracted version of the reference genome and the extracted location. The system generates a report summarizing all the contextual information associated with a node of the graph-based reference genome, and provides the generated report to a user.

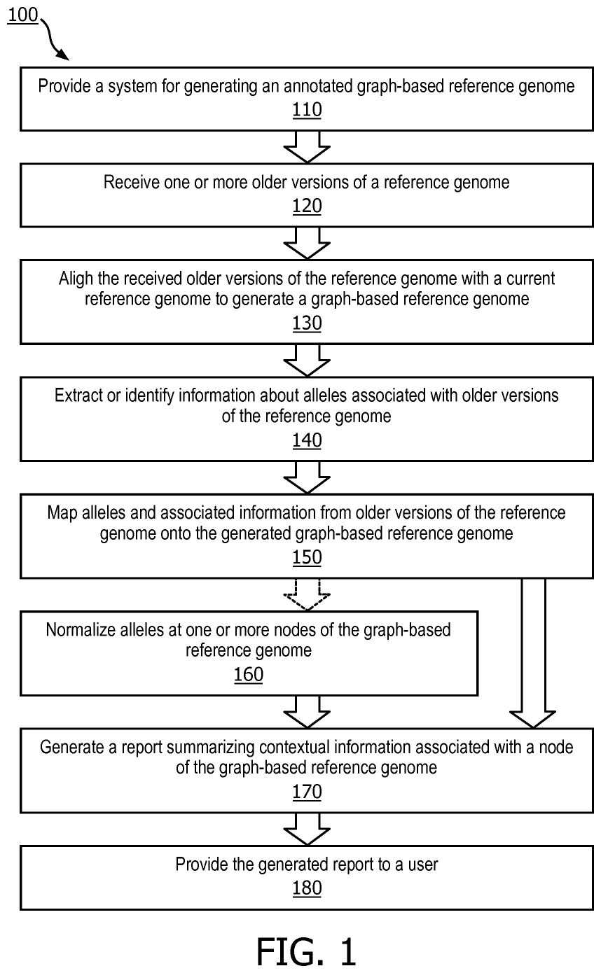

Referring to FIG. 1, in one embodiment, is a flowchart of a method 100 for generating an annotated graph-based reference genome. At step 110, a system for generating an annotated graph-based reference genome is provided. The system may be any of the systems described or otherwise envisioned herein, and may comprise any of the components or modules described or otherwise envisioned herein.

At step 120 of the method, one or more previous versions of a reference genome are received by the system or provided to the system. Each of these previous versions includes a plurality of nodes, at least some of the these nodes comprising information identifying the version of the reference genome the node came from, as well as a location within that version of the reference genome where the node is located. According to an embodiment, a node represents a SNP, mutation, allele, and/or k-mer of length k.

The reference genome can be a human reference genome, or a reference genome from any other organism. The previous versions of the reference genome can be obtained or received from any source, including but not limited to a database of previous versions. For example, one or more versions of a reference genome may be privately or publicly available for use, and may be stored in a private or public repository or database for retrieval. Typically a reference genome is digital and can be stored in a database, and can be communicated electronically via a wired and/or wireless communication system from the database to the annotated graph-based reference genome generation system.

Typically, differences between versions of a reference genome include more reliable data for specific locations, changes to the coordinates or location of certain sequences, new information about prior gaps in the sequence, and many other differences. One of the biggest differences relevant to the present disclosure is the modification of coordinates of a sequence. For example, sequence k (which may be a single nucleotide or SNP or may be a sequence of nucleotides) on chromosome 5 may be located at a first position in a first version of a reference genome, but additional sequencing and analysis may reveal that sequence k is more properly positioned at a second location on chromosome 5. Accordingly, a subsequent version of the reference genome will move sequence k to the second location. The previous version of the reference genome, and the published literature discussing sequence k, will still have sequence k located at the first location on chromosome 5.

At step 130 of the method, each of the received older versions of the reference genome is aligned with a current reference genome to generate a graph-based reference genome. This alignment is based at least in part on the location information from the nodes of the received older version of the reference genome. Since the nodes of the received older versions of the reference genome comprise location information, this location information can be utilized to identify where, in the current version of the reference genome, that location can be found. In some cases the coordinates of the location will not have changed, while in many cases the coordinates of the location will have changed significantly.

According to an embodiment, the system comprises or is in communication with a comparative system or module that comprises or provides information about where locations in previous versions of the reference genome can be found in the current version of the reference genome. For example, within the system the current version of the reference genome may contain at a plurality of nodes information about where that node was located in previous versions of the reference genome. Additionally or alternatively, the previous versions of the reference genome may be annotated with or otherwise comprise information about where nodes from that version of the reference genome can be found in the current version of the reference genome.

For example, the current version of the human reference genome released from the Genome Reference Consortium in 2013 is GRCh38, sometimes called build 38, although modifications of GRCh38 have been subsequently released. Accordingly, any of the previous versions or builds may be mapped onto GRCh38 using the methods described or otherwise envisioned herein. In the future a new version such as GRCh39 may be released and previous versions or builds can be mapped onto GRCh39. The methods and systems described herein function regardless of which version or build is utilized as the current version of the human reference genome. Additionally, the methods and systems described herein function for any organism having a reference genome with multiple versions or builds.

In the past, scientific literature examining an aspect of human genetics used one or more versions of the human genome released prior to the current version GRCh38. Accordingly, the scientific literature will typically reference the specific version of the human reference genome used for the analysis or study. However, in cases where the scientific literature fails to reference the specific version of the human reference genome used for the analysis or study, the date of the publication and/or the research (which can be gleaned or deduced from the publication citation or publication metadata) can be utilized to infer which version of the human reference genome was likely used for the analysis or study.

According to an embodiment, to express information for a strand, and to thus distinguish between reading DNA in forward or reverse, the graph-based reference genome can be constructed in a bi-directional method or format. Several methodologies are available to build the graph-based reference genome, including multiple genome alignment based on phylogenetic tree, De Bruijn graph construction, and many other methods. For example, when used for genome assembly, De Bruijn graphs typically comprise a node representing a k-mer with directed edges representing an overlap of k−1 bases between two nodes, although many other variations are possible, as are many other methods of graph construction.

According to an embodiment, the method may use all prior versions of a reference genome, including any patches or other modifications, and any accumulated polymorphisms, as input during construction ofthe graph-based reference genome. According to another embodiment, the method may only use some prior versions of a reference genome as input during construction of the graph-based reference genome.

According to an embodiment, for each allele from a previous version of a reference genome aligned to the current version of the reference genome, a data structure can be constructed or utilize to mark which version of the reference genome included the allele, and the coordinates of the allele in that version of the reference genome, including chromosome number and location. Accordingly, a plurality of nodes or alleles of the current version of the reference genome will comprise information about that node or allele in some or all previous versions of the reference genome utilized to generate the graph-based reference genome.

At step 140 of the method, the system extracts, identifies, and/or receives information about one or more alleles from scientific literature. For example, the system may comprise or have access to a corpus of literature and references, which may be public and/or private databases. There are currently many different databases of scientific literature, and any of these databases may be utilized. From this corpus of literature and references, information about an allele can be identified and/or extracted. Together with an identification of the allele, other information can be identified and/or extracted, including but not limited to: (1) a reference SNP cluster ID number or other accession number identifying the allele; (2) coordinates for the allele, including chromosome number and location; (3) the reference genome utilized for the coordinates; and/or (4) contextual information about the allele.

According to an embodiment the contextual information may include, for example, medical or trait information identified as being associated or affected by the allele, polymorphisms identified for the allele, populations associated with the allele, research information about the allele, citation information for the allele, and/or any other information about the allele, the reference, and/or the research.

According to an embodiment, allele information can be reported in the literature in a structured and/or unstructured format. Structured formats are more easily aligned onto the graph-based reference genome. However, for unstructured information, an explicit ETL (Extracting, Transforming and Loading) process can be utilized. The system may comprise a synonym table to account for the various names utilized for prior versions of a reference genome. For example, hg19 and GRCH37 refer to the same prior version o f the human reference genome. The system may also comprise a module or algorithm configured or designed to extract relevant mutation/allele information as tuples, such as the reference identification, chromosome number, coordinates, reference and alternative alleles, strand information, somatic/germline, sequencing modality (such as microarray, WGS, or WES), phenotype(s), diagnosis, anatomic locations, age, gender, race, medical history, and/or patient ID, among other possible information. According to an embodiment, the information is parsed via medical ontology based natural language processing pipelines. Relationships between an allele, a phenotype, metadata, and any other information can be saved in a data structure such as an RDBMS (relational database management system), among other possible data structures.

According to an embodiment, this step and other steps of the method will necessarily comprise heavily computational work. For example, this step may comprise a review of thousands or millions of pieces of literature, including summarizing all relevant information. Methods or systems may be implemented to facilitate the computational work. For example, an infrastructure setup via Hadoop/MapReduce may address the needs in whole or in part. Many other methods and systems can be utilized to facilitate this computationally intensive analysis.

At step 150 of the method, the system maps the extracted, received, or identified allele and associated contextual information onto a node of the graph-based reference genome. The mapping is based at least in part on the location of the extracted allele within the older version of the reference genome. For example, an allele from a prior version of the reference genome may be mapped to a node of the graph-based reference genome. Along with the allele, the contextual information associated with the allele can be mapped to the node, including any or all of the contextual information disclosed or otherwise envisioned herein. The mapping is based at least in part on location information associated with the extracted, received, or identified allele, and can be cross-referenced to location information for the graph-based reference genome. According to an embodiment, an allele may have multiple corresponding coordinates from one or more prior versions of the reference genome. The system can review each of them and query the RDBMS during mapping.

At optional step 160 of the method, the system normalizes a plurality of alleles or results associated with a node of the graph-based reference genome. According to an embodiment, many of the reported alleles are not mutations but are normal polymorphisms, and normalization will identify these normal polymorphisms. Any method for normalization can be utilized.

At step 170 of the method, the system generates a report summarizing all the contextual information associated with a node of the graph-based reference genome. The system can do this for one node or multiple nodes. According to an embodiment, the system can query the RDBMS or other data structure for information about a node, an allele, a location in the graph-based reference genome, and/or a location in a prior version of the reference genome. The results can be summarized across different genome versions into one or more categories including: allele frequency, appearance times, surrounding mutation rate, co-mutation rate, phenotype groups, and/or any other information.

At step 180 of the method, the system provides the generated report to a user, via a user interface of the system. The report can comprise any format, and is preferably a format which is easy to review and interpret. The report can be provided via any mechanism, including but not limited to a display, readout, download, upload, printout, email, and many other processes.

According to an embodiment, the generation and use of a graph-based reference genome is a significant improvement over prior reference genome formats, and solves many long-felt problems in the art. For example, few genomic regions are annotated with accumulated clinical and/or biological knowledge for most biomedical research and applications. To explain an unknown genomic area, an open learning framework has to be put into place for mutation-oriented knowledge accumulation. For example, if unknown somatic mutations are detected in a cancer patient, prioritizing those mutations can influence downstream clinical decision-making. One method for prioritization is to examine each mutation's allele frequency and how many times the mutation has been reported, although this is an inefficient and unguided method of analysis. Summary of an allele from the literature, in the context of a graph-based reference genome, provides much more valuable and actionable information. Accordingly, the methods and systems disclosed herein can significantly improve patient care and outcomes compared to prior reference genome methods and systems. According to another embodiment, the data assembled from the corpus of literature and mapped onto the graph-based reference genome could also facilitate the biomarker discovery process.

According to another embodiment, a graph-based reference genome infrastructure can allow third-party entities such as biopharmaceutical companies or diagnosis companies to maintain proprietary mutation-phenotype databases regardless of how the reference genome evolves. For example, a customer may have mutations that are detected but refer to different versions of the reference genome, such as hg18 or hg19. These mutations can be accommodated onto the graph-based reference genome. For example, if a user queries specific genome coordinates in reference to a specific prior version of the reference genome, the information associated with those coordinates can be extracted from the graph-based reference genome regardless of which version of the reference genome is being utilized or referred to.

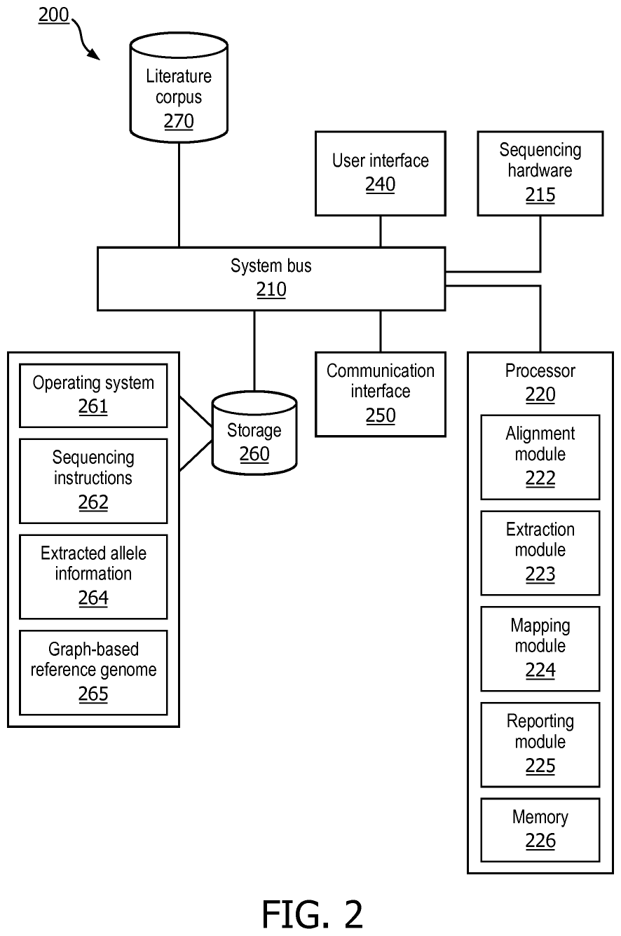

Referring to FIG. 2 is a schematic representation 200 of a system and method for generating an annotated graph-based reference genome as described or otherwise envisioned herein. System 200 includes one or more of a processor 220, memory 226, user interface 240, communications interface 250, and storage 260, interconnected via one or more system buses 210. In some embodiments, such as those where the system comprises or implements a sequencer or sequencing platform, the hardware may include additional sequencing hardware 215, which may be any sequencer or sequencing platform. It will be understood that FIG. 2 constitutes, in some respects, an abstraction and that the actual organization of the components of the system 400 may be different and more complex than illustrated.

According to an embodiment, system 200 comprises a processor 220 capable of executing instructions stored in memory 226 or storage 260 or otherwise processing data. Processor 220 performs one or more steps of the method, and may comprise one or more of the modules described or otherwise envisioned herein. Processor 220 may be formed of one or multiple modules, and can comprise, for example, a memory 226. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 226 can take any suitable form, including a non-volatile memory and/or RAM. The memory 226 may include various memories such as, for example a cache or system memory. As such, the memory 226 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user such as an administrator. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200. Where system 200 implements a sequencer and includes sequencing hardware 215, storage 260 may include sequencing instructions 262 for operating the sequencing hardware 215. According to an embodiment, storage 260 may include an extracted allele database 464 generated or populated pursuant to the methods described or otherwise envisioned herein. According to an embodiment, storage 260 may include a graph-based reference genome 265 generated pursuant to the methods described or otherwise envisioned herein.

It will be apparent that various information described as stored in storage 260 may be additionally or alternatively stored in memory 226. In this respect, memory 226 may also be considered to constitute a storage device and storage 460 may be considered a memory. Various other arrangements will be apparent. Further, memory 226 and storage 260 may both be considered to be non-transitory machine-readable media. As used herein, the term non-transitory will be understood to exclude transitory signals but to include all forms of storage, including both volatile and non-volatile memories.

System 200 may also comprise a corpus of literature 270. This corpus may be a single database or multiple databases. The database may be a component of system 200, or system 200 may be in communication or otherwise access the corpus of literature 270. The database may comprise a plurality of articles, papers, posters, abstracts, or other information, which may be obtained or found in private and/or public sources.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where system 200 is implemented in a cloud computing system, the various hardware components may belong to separate physical systems. For example, processor 220 may include a first processor in a first server and a second processor in a second server. Many other variations and configurations are possible.

According to an embodiment, processor 220 comprises one or more modules to carry out one or more functions or steps of the methods described or otherwise envisioned herein. For example, processor 220 may comprise an alignment module 222, an extraction module 223, a mapping module 224, and/or a reporting module 225.

According to an embodiment, alignment module 222 aligns or facilitates alignment of a received or identified older version of a reference genome with a current reference genome to generate a graph-based reference genome. This alignment can be based at least in part on the location information from nodes of the received older version of the reference genome. Since the nodes of the received older versions of the reference genome comprise location information, this location information can be utilized to identify where, in the current version of the reference genome, that location can be found. In some cases the coordinates of the location will not have changed, while in many cases the coordinates of the location will have changed significantly. According to an embodiment, alignment module 222 comprises or provides information about where locations in previous versions of the reference genome can be found in the current version of the reference genome.

According to an embodiment, extraction module 223 extracts, identifies, and/or receives information about one or more alleles from scientific literature found in the corpus of literature 270. The extracted allele information 264 can be stored, for example, in storage 260 or in a variety of other locations or databases. Together with an identification of the allele, other information can be identified and/or extracted, including but not limited to: (1) a reference SNP cluster ID number or other accession number identifying the allele; (2) coordinates for the allele, including chromosome number and location; (3) the reference genome utilized for the coordinates; and/or (4) contextual information about the allele. According to an embodiment the contextual information may include, for example, medical or trait information identified as being associated or affected by the allele, polymorphisms identified for the allele, populations associated with the allele, research information about the allele, citation information for the allele, and/or any other information about the allele, the reference, and/or the research.

According to an embodiment, mapping module 224 maps the extracted, received, or identified allele and associated contextual information onto a node of the graph-based reference genome 265. The mapping is based at least in part on the location of the extracted allele within the older version of the reference genome. For example, an allele from a prior version of the reference genome may be mapped to a node of the graph-based reference genome. Along with the allele, the contextual information associated with the allele can be mapped to the node, including any or all of the contextual information disclosed or otherwise envisioned herein. The mapping is based at least in part on location information associated with the extracted, received, or identified allele, and can be cross-referenced to location information for the graph-based reference genome. According to an embodiment, an allele may have multiple corresponding coordinates from one or more prior versions of the reference genome. The system can review each of them and query the RDBMS during mapping.

According to an embodiment, reporting module 225 system generates a report summarizing all the contextual information associated with a node of the graph-based reference genome. The module can do this for one node or multiple nodes. According to an embodiment, the module can query the RDBMS or other data structure for information about a node, an allele, a location in the graph-based reference genome, and/or a location in a prior version ofthe reference genome. The results can be summarized across different genome versions into one or more categories including: allele frequency, appearance times, surrounding mutation rate, co-mutation rate, phenotype groups, and/or any other information. According to an embodiment, reporting module 225 also provides or directs the system to provide the generated report to a user, via a user interface of the system.

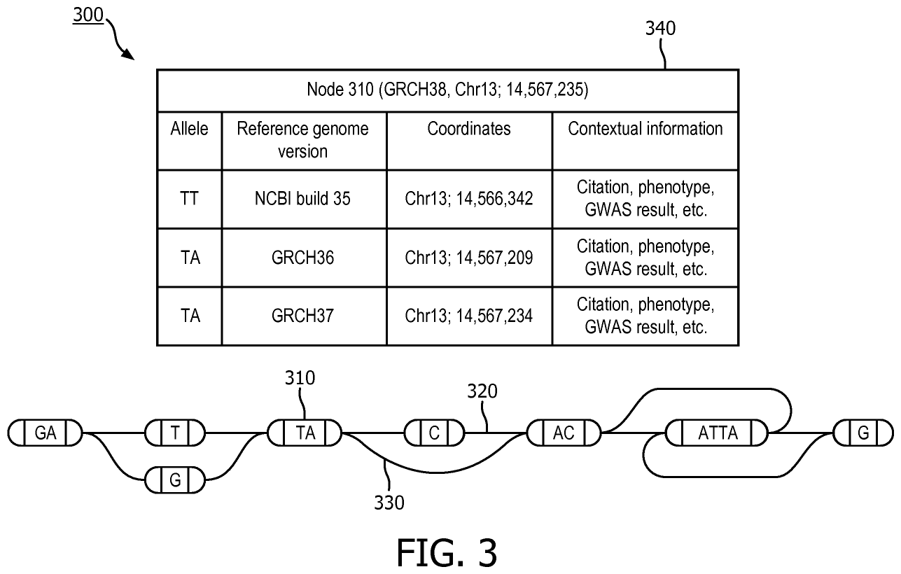

According to an embodiment is a graph-based reference genome as described or otherwise envisioned herein. Referring to FIG. 3, in one embodiment, is a graph-based reference genome 300 based on a current version of a reference genome, and encoding information from a plurality of different versions of the reference genome. Graph-based reference genome 300 comprises, for example, a plurality of nodes 310 which can be labeled, identified or otherwise annotated with sequences, allele information, and/or contextual information as described or otherwise envisioned herein. Graph-based reference genome 300 also comprises, for example, a plurality of edges 320 which connect two nodes via either of their respective ends. The graph-based reference genome 300 can also include paths 330, which connect two nodes via either of their respective ends but provide alternative sequencing, coordinates, or other modifications. For example, paths can provide coordinate systems relative to genomes encoded in the graph, thereby allowing stable mappings to be produced even if the structure of the graph is changed.

According to an embodiment, a plurality of nodes 310 of the graph-based reference genome comprise information from one or more previous versions of the reference genome. The information may include, for example, an allele, an identification of the reference genome from which the allele was extracted or identified, information about the coordinates of the allele in that reference genome, and/or contextual information, among other possible information. Referring to FIG. 3, for example, is a table or data structure 340 associated with node 310. The node may be directly annotated with the information in table or data structure 340, or node 310 may be associated in memory with table or data structure 340, and/or node 310 may comprise a pointer or other link to table or data structure 340. Although the table shows three prior versions of the reference genome, the table may comprise information about one, several, or all prior versions of the reference genome.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for generating an annotated graph-based reference genome, comprising:
receiving one or more versions of a reference genome, being older versions of a current reference genome, each of the one or more versions of the reference genome comprising a plurality of nodes, at least some of which comprise information identifying the version of the reference genome and a location within that version of the reference genome for the respective node;
aligning each of the one or more received older versions of the reference genome to the current reference genome to generate a graph-based reference genome, wherein the alignment is based at least in part on the location information from the nodes of the received older version of the reference genome;
extracting, from a corpus of references at least some of which each comprise information about an allele and contextual information associated with that allele, an allele and contextual information associated with the allele, wherein the respective reference identifies: (i) one of the one or more received older versions of the reference genome, and (ii) a location of the allele within the identified older version of the reference genome; and
mapping the extracted allele and associated contextual information onto a node of the graph-based reference genome, based on the identified older version of the reference genome and the location of the extracted allele within that identified older version of the reference genome.

2. The method of claim 1, further comprising:
generating a report summarizing all the contextual information associated with a node of the graph-based reference genome; and
providing, via a user interface, the generated report to a user.

3. The method of claim 2, wherein the report comprises one or more of an allele frequency, appearance information, surrounding mutation information, and/or co-mutation rate.

4. The method of claim 1, wherein mapping comprises annotating the node with the extracted allele and associated contextual information.

5. The method of claim 1, wherein mapping comprises annotating the node with an identification of the reference from which the allele was extracted.

6. The method of claim 1, wherein the contextual information comprises information about a trait or medical condition associated with the allele.

7. The method of claim 1, wherein the contextual information comprises an identification of a reference from which the allele was identified or extracted.

8. The method of claim 1, wherein the contextual information comprises information about one or more people in which the allele was identified.

9. The method of claim 1, further comprising normalizing a plurality of alleles associated with a node of the graph-based reference genome.

10. A system for generating an annotated graph-based reference genome, comprising:
an alignment module configured to align each of a plurality of received older versions of a reference genome to a current reference genome to generate a graph-based reference genome, wherein the alignment is based at least in part on information from nodes of the received older version of the reference genome, at least some of the nodes comprising information identifying the version of the reference genome and a location within that version of the reference genome for the respective node;
an extraction module configured to extract, from a corpus of references at least some of which each comprise information about an allele and contextual information associated with that allele, an allele and contextual information associated with the allele, wherein the respective reference identifies: (i) one of ihe one or more received older versions of the reference genome, and (ii) a location of the allele within the identified older version of the referenced genome;
a mapping module configured to map a plurality of identified alleles onto one or more nodes of the graph-based reference genome based on the identified older version of the reference genome and the location of the extracted allele within that identified older version of the reference genome, wherein each of the plurality of identified alleles also comprises contextual information which is mapped onto the respective node with the respective allele;
a reporting module configured to generate a report summarizing all the contextual information associated with a node of the graph-based reference genome; and
a user interface configured to provide the generated report to a user.

11. (canceled)

12. The system of claim 10, wherein the contextual information comprises information about a trait or medical condition associated with the allele.

13. The system of claim 10, wherein the contextual information comprises an identification of a reference from which the allele was identified or extracted.

14. (canceled)

15. (canceled)

16. An annotated graph-based reference genome, generated in accordance with the method of claim 1.
